(19) United States
(12) Patent Application Publication
(10) Pub. No.: US 2004/0116991 A1
(43) Pub. Date: Jun. 17, 2004

(54) Implantable electrode structure

(76) Inventor: Thierry Herve, Le Sappey En Chartreuse (FR)

Correspondence Address: DUANE MORRIS LLP, 100 COLLEGE ROAD WEST, SUITE 100, PRINCETON, NJ, 08540-6604, US

(21) Appl. No.: 10/468,696

(22) PCT Filed: Feb. 28, 2002
(86) PCT No.: PCT/FR02/00732

(30) Foreign Application Priority Data
Feb. 28, 2001 (FR) 01-02727

(51) Int. Cl. A61N 1/05

(52) U.S. Cl. 607/116

(57) ABSTRACT

The invention concerns a structure comprising a flexible insulating support film (1) and electrodes (3) capable of measuring the electrical activity of a physiological medium or of stimulating it. The structure comprises, on the side of the electrodes, at least an element (18, 20, 22, 35, 38) made of insulating material projecting relative to the surface of the structure.

The present invention relates to a structure supporting electrodes likely to detect the electric activity of an organ, like a nerve or the heart.

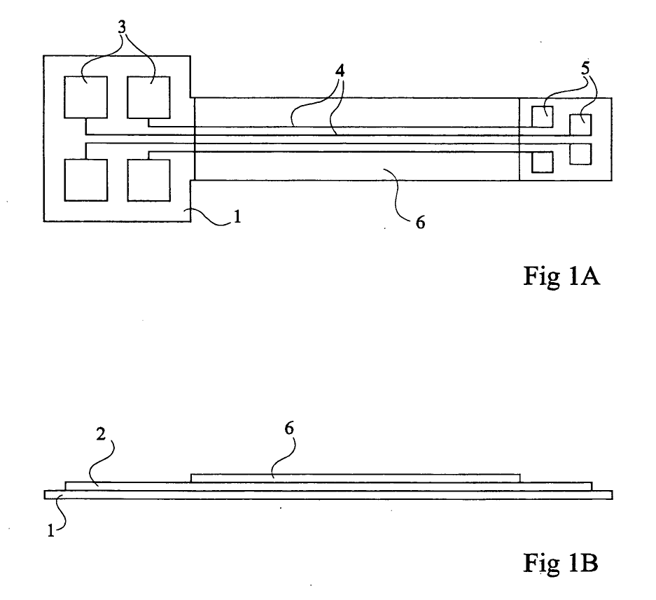

FIG. 1A describes a known structure of this type. FIG. 1B shows the structure of FIG. 1A in cross-section. The structure comprises a support film 1 made of a flexible isolating material like polyimide. On the film is deposited a conductive layer 2 etched to define electrodes 3, connected by conductive tracks 4 to connection pads 5. Except for the electrodes and the pads, the assembly is covered with an isolating film 6. The obtained structure is flexible and has a very small thickness, on the order of a few micrometers. Typically, electrodes 3 have a size of a few micrometers, and the structure has a length of a few centimeters.

When used, the structure is inserted on or in the vicinity of the organ, the electric activity of which must be measured. Upon insertion of the structure, the handling of the structure is uneasy due to its flexibility. If the structure is handled with pliers, it may be damaged. As it is being inserted, it may deform and uneasily reach the desired location. Further, once in place, the prior art structure uneasily keeps any determined shape. It has a tendency to sliding and to moving away from the position where it has been placed.

An object of the present invention is to form an electrode structure of the above-mentioned type enabling easy handling and eased insertion.

Another object of the present invention is to form such a structure easily keeping any desired shape.

Another object of the present invention is to form a structure which does not slide from the location where it has been placed.

To achieve these and other objects, the present invention provides a structure comprising a flexible isolating support film and electrodes capable of measuring the electric activity of a physiological medium or of stimulating it, the assembly formed by said film and said electrodes having a thickness smaller than 50 micrometers. The structure comprises, on the electrode side, at least one element of isolating matter protruding from the surface of the structure, the thickness of said element being equal to at least 20 micrometers.

According to an embodiment of the present invention, the electrodes comprise holes crossing the structure.

According to an embodiment of the present invention, the protruding element has the shape of a vertical toe capable of cooperating with an opening of the structure.

According to an embodiment of the present invention, the protruding element has the shape of a continuous or discontinuous ring and surrounds the electrodes.

According to an embodiment of the present invention, the protruding element has the shape of several vertical toes placed at the level of the structure portion which comprises the electrodes.

According to an embodiment of the present invention, the protruding element has the shape of a rigid longitudinal bar.

According to an embodiment of the present invention, the protruding element has the shape of a lateral toe.

According to an embodiment of the present invention, the structure comprises several protruding elements of variable number and distribution, so that the rigidity of the structure is controlled.

According to an embodiment of the present invention, the structure comprises openings in its isolating portion.

The present invention also provides an assembly comprising the above structure and a rigid and/or deformable device, the structure being immovably attached to said device.

The foregoing objects, features, and advantages of the present invention will be discussed in detail in the following non-limiting description of specific embodiments in connection with the accompanying drawings, in which:

FIGS. 1A and 1B, previously described, show electrode structures of prior art;

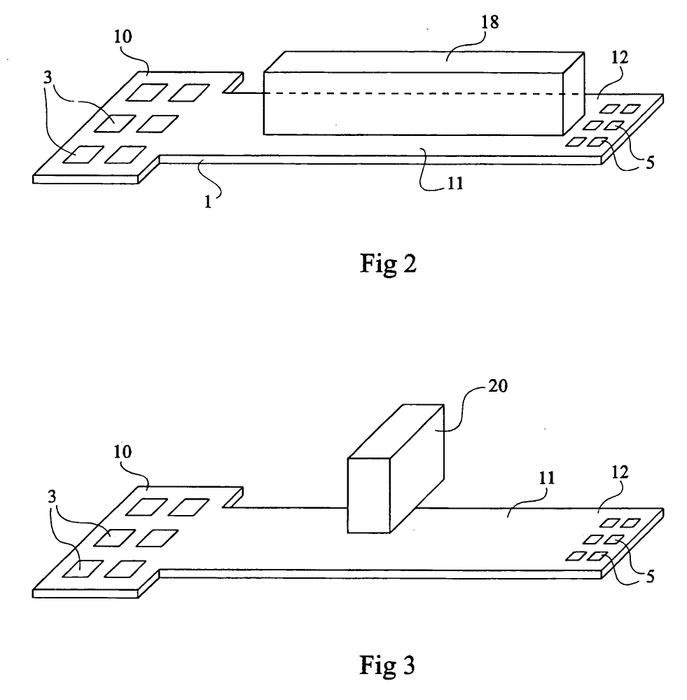

FIG. 2 shows a first embodiment of the present invention;

FIG. 3 shows a second embodiment of the present invention;

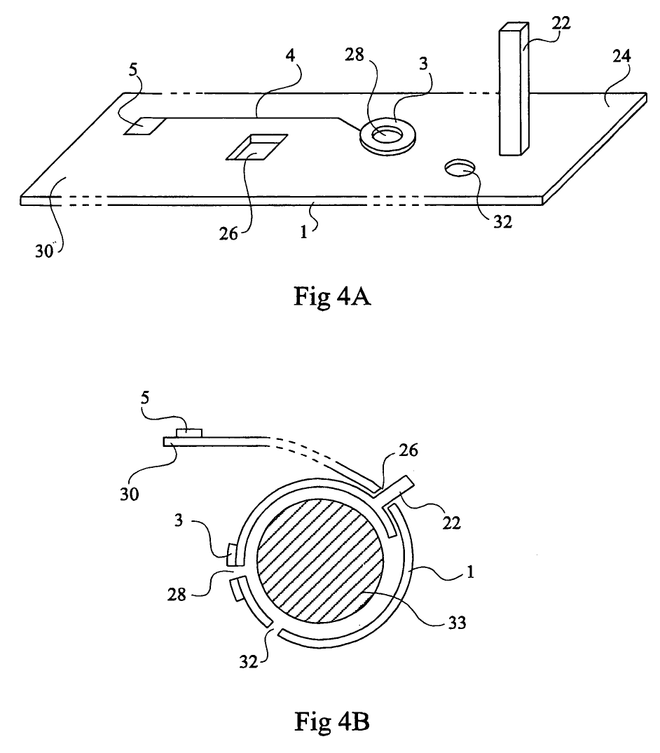

FIGS. 4A and 4B respectively show a third embodiment of the present invention and a possible use thereof;

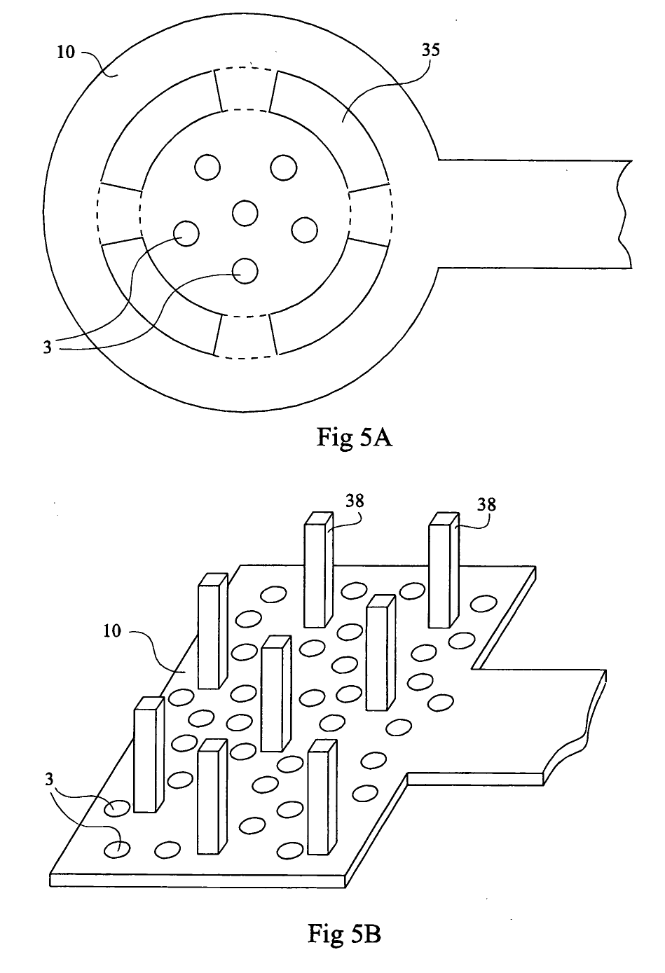

FIGS. 5A and 5B respectively show a fourth and a fifth embodiment of the present invention; and

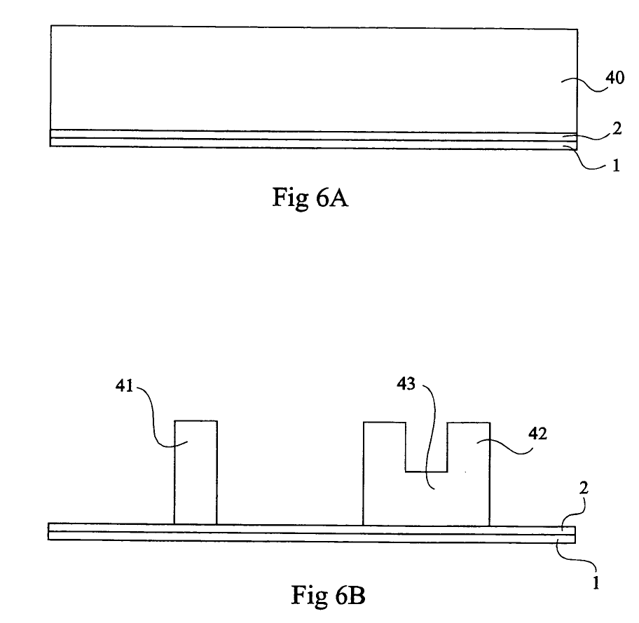

FIGS. 6A and 6B illustrate steps enabling forming of structures according to the present invention.

In the drawings, same reference numerals represent identical elements or elements having the same function. The scales have not been respected, especially as concerns the vertical dimension.

In FIG. 2, a film 1 of a structure according to a first embodiment of the present invention comprises a head portion 10 supporting electrodes 3, an intermediary portion 11, and a terminal portion 12 supporting pads 5. For simplicity, the tracks of connection between the electrodes and the pads have not been shown. On intermediary portion 11 is a parallelepiped-shaped longitudinal bar 18. Bar 18 represents a tri-dimensional element forming a prominent relief with respect to the structure surface, on the same side as the electrodes. Bar 18 has a variable height with respect to the thickness of the support film and of the conductive layer forming the electrodes. While the support film, as well as the layer forming the electrodes, has a typical thickness of a few micrometers only, the height of bar 18 may range from approximately 20 micrometers to 500 micrometers or more. In a practical example of implementation, the height of bar 18 has been chosen to be equal to 100 micrometers.

Bar 18 makes intermediary portion 11 more rigid and may be used as a guide. For example, it may insert in a slideway of a rigid element used to insert the structure. Bar 18 may also be attached to the end of any device, for example, of endoscope or stereostatic type. Thus, the structure may be placed with a submillimetric precision. This precision of the structure placing, possible with the present invention, may have a major advantage in certain cases, like when the structure is placed in the brain. Bar 18 may also be attached to a device intended to remain in the body. Bar 18 may also be held by pliers, thus easing the structure insertion or its handling during intervention.

In an alternative implementation not shown, bar 18 is reduced to a mere rectangle parallelepiped of square cross-section and forms a vertical toe arranged for example at the center or towards the front of the structure. This toe may be seized by pliers and ease the structure handling.

FIG. 3 shows a second embodiment of the present invention. The structure of FIG. 3 is largely similar to that of FIG. 2. It also comprises a three-dimensional element protruding out of the structure surface. Here, the protruding element has the shape of a lateral toe 20 located at the level of intermediary portion 11 of the structure. Toe 20 has a height which, like bar 18 of FIG. 2, ranges between approximately 20 micrometers and 500 micrometers or more.

Toe 20 may be used for the handling and may easily be seized by pliers. Toe 20 may be used for other purposes than the handling. For example, it may be used as a stop for a slideway on which the rear portion of the structure is inserted. Toe 20 can thus cooperate with bar 18 of FIG. 2, and stop in translation a slideway in which bar 18 is inserted. In FIG. 3, toe 20 is arranged at the level of intermediary portion 11 of the structure. Of course, it may be at the level of any portion of the structure, for example, at the level of head portion 10.

FIG. 4A shows another embodiment of a structure according to the present invention. As in the previous embodiments, the structure comprises a protruding element, here in the form of a vertical toe 22, of parallelepipedal shape and with a height ranging between approximately 20 micrometers and 500 micrometers or more. Toe 22 is located close to an end 24 of the structure. The structure also comprises an opening 26 thoroughly crossing the structure. Opening 26 has a size such that toe 22 can insert into opening 26. Between opening 26 and toe 22 are arranged electrodes 3, only one of which is shown. Each electrode 3 is bored with a thorough hole 28 and is connected by a conductive track 4 to a pad 5. Pads 5 are located at one end 30 of the structure, opposite to end 24. The structure may also comprise thorough holes 32 (only one of which is shown) between opening 26 and toe 22.

FIG. 4B shows a mode of possible use of the structure of FIG. 4A. In FIG. 4B, the hatched portion shows a nerve 33 in cross-section. The structure is wound around the nerve and toe 22 is inserted into opening 26. The distance between opening 26 and toe 22 is chosen so that the structure does not hold the nerve too tight while enabling adequate measurement of its activity. Electrodes 3 are arranged around the nerve. Since the electrodes are located on the surface of film 1 which does not contact the nerve, the hole 28 through each electrode enables the electric signal to pass from the nerve onto the electrode. Other holes 32 may be located in the isolating part of the structure located between opening 26 and toe 22. Their function is to let the physiological liquid through and enable an equilibrium of this liquid on either side of the structure. Pads 5, at end 30, enable connection to an outer circuit.

It should be noted that holes 28 of the electrodes are also used for the irrigation, like holes 32, and that holes 32 also favor the electric conduction. The irrigation function due to holes 28 and 32 is very important in certain applications. For example, in the above application, a non-irrigated nerve looses its functionality very fast, which is not desirable.

It should also be noted that, due to the cooperation of toe 22 and of opening 26, the structure is well attached around the nerve and does not slide. Several openings 26 may be made in the structure, to adapt the structure to different sizes of nerves or organs around which it is inserted.

In an alternative of this embodiment, several toes 22 arranged at intervals that may be regular or not may insert into an opening 26 of the structure. The structure can thus take a complex shape particularly stable in time, for example, the shape of a cylinder, of a spiral or of a helix.

In an alternative of this embodiment, the structure comprises one or several toes 22 intended to insert into openings of a rigid device external to the structure. The external device may be planar or not and may have any determined shape.

The structure may also be immovably attached to a rigid and/or deformable device. The rigid and/or deformable device may be formed by means of shape-memory materials and may be adapted to the specific concavity of an organ, for example, the cochlea.

FIG. 5A shows another embodiment of the present invention. In FIG. 5A, only the head portion 10 of the structure is shown, here in the form of a disk. Electrodes 3, here of circular shape, are arranged in the central portion of portion 10. Electrodes 3 are surrounded with a protruding ring-shaped element 35. Element 35 may form a continuous ring or a discontinuous ring, to avoid increasing the rigidity of portion 10 too much. The height of element 35 is of the same order of magnitude as the height of the protruding elements of the previous embodiments. Element 35 forms a rim which opposes the sliding of portion 10 on the surface where it is applied.

Also, in many applications, a conductive paste or gel intended to favor the electric contact between the electrodes and the organ, for example, the skin, is used. In prior art, the presence of a gel favors the sliding of the structure on the organ. Also, the gel may come out of the structure and no longer fulfill its function. In the present invention, element 35, which forms a sort of basin, may be used as a receptacle for the gel, which is retained, even in cases where element 35 is discontinuous.

FIG. 5B shows another embodiment of the present invention. The structure of FIG. 5B exhibits the same advantages as the structure of FIG. 5A. In FIG. 5B, head portion 10 comprises a relatively large number of toes 38, of a shape similar to that of toes 22 of FIG. 4A. Toes 38 enable, due to the asperity that they represent, good catching with the surface on which they are applied. When a conductive paste or gel is placed on portion 10, toes 38 retain the gel or paste, which do not leave the surface of portion 10 of the structure. In FIG. 5B, electrodes 3 are distributed in substantially uniform fashion between toes 38.

Another advantage of elements 35 and 38 is that the structures comprising them may easily be inserted in a garment or an undergarment. The garment and its position on the body must enable passing of electric signals from the tested organ, for example, the skin, to the structure electrodes. For example, the garment will be open or conductive at the electrode level, and in contact with the skin. Due to elements 35, 38, the structure is particularly well attached to the fibers of the fabric forming the garment and remains in a stable position. Such a structure, inserted in a garment, enables continuous measurements, like a heart monitoring.

In an embodiment not shown of the structure, protruding elements simply aim at rigidifying the structure. For example, a series of toes of the type of toe 22 are arranged along intermediary portion 11, the rigidity of the structure being a function of the number and of the distribution of the toes. Thus, the fact of arranging protruding elements enables local control of the structure rigidity. This is particularly advantageous in cases where, once in place, the structure must exhibit portions of different curvature. Thus, the rectilinear portions of the structure will advantageously be relatively rigid, while the curved portions will have a lesser rigidity.

A method enabling forming structures according to the present invention will now be described in relation with FIGS. 6A and 6B.

In FIG. 6A, a support film 1 made of an isolating flexible material, for example, polyimide, is covered with a conductive layer 2. The assembly formed by film 1 and layer 2 has a small thickness, generally under 50 micrometers. Conductive layer 2 is properly etched to expose electrodes connected to contact pads enabling connection to an outer circuit. On layer 2 is deposited a thick layer 40 of an isolating material, for example, the same material as layer 1 or a polymer selectively etchable with respect to the material of FIG. 1. Layer 40 is deposited in one go or in several goes. The thickness of layer 40 is not critical. It may range from approximately, 20 micrometers to 500 micrometers or more. Then, layer 40 is etched, for example, by plasma.

FIG. 6B shows a possible example of a structure resulting from the etching of layer 40. Layer 40 has been etched totally in places and incompletely in other places. In the resulting structure, layers 1 and 2 remain unchanged, and there remains, at the structure surface, two protruding tridimensional elements 41 and 42. Element 41 has a parallelepipedal shape. It may for example appear in the form of a vertical bar extending over the entire structure thickness, or in the form of a toe having, for example, a square cross-section. Element 42 has a crenel shape and exhibits an incompletely-etched portion 43.

Of course, the present invention is likely to have various alterations, modifications, and improvements which will readily appear to those skilled in the art. In particular, the embodiments described in FIGS. 2, 3, 4A, 5A, and 5B are examples only and any structure with electrodes supporting, on the electrode side, protruding elements at its surface, is within the field of the present invention, whatever the specific function of the protruding elements.

Elements 3, as well as the protruding elements of the structure, may be arranged anywhere on the structure surface.

Holes 32 described in relation with FIGS. 4A and 4B may be present on any structure according to the present invention and at any location. Similarly, electrodes 3 may exhibit holes 28 in any structure according to the present invention.

Although the structure of the present invention has been described as used to detect the electric activity of an organ, any application of the structure is within the field of the present invention, like the stimulating of an organ by means of electric signals provided by electrodes 3.

It should be noted that the structure of the present invention may comprise an additional isolating layer covering the assembly formed of film 1 and the conductive layer forming the electrodes, the pads, and the tracks therebetween. In this case, the additional isolating layer is a thin flexible layer that may have the same thickness as film 1 and does not cover the structure at the location of the electrodes and the pads. This additional isolating layer corresponds to layer 6 of prior art. It may result from the deposition of a thin layer on the structure, or of an incomplete etching of thick layer 40.

1. A structure comprising a flexible isolating support film (1) and electrodes (3) capable of measuring the electric activity of a physiological medium or of stimulating it, the assembly formed by said film and said electrodes having a thickness smaller than 50 micrometers, characterized in that it comprises, on the electrode side, at least one element (18, 20, 22, 35, 38) of isolating matter protruding from the surface of the structure, the thickness of said element being equal to at least 20 micrometers.

2. The structure of claim 1, wherein the electrodes (3) comprise holes (28) running through the structure.

3. The structure of any of claims 1 and 2, wherein the protruding element has the shape of a vertical toe (22) capable of cooperating with an opening (26) of the structure.

4. The structure of any of claims 1 and 2, wherein the protruding element has the shape of a continuous or discontinuous ring (35) and surrounds the electrodes.

5. The structure of any of claims 1 and 2, wherein the protruding element has the shape of several vertical toes (38) placed at the level of the structure portion which comprises the electrodes.

6. The structure of any of claims 1 and 2, wherein the protruding element has the shape of a rigid longitudinal bar (18).

7. The structure of any of claims 1 and 2, wherein the protruding element has the shape of a lateral toe (20).

8. The structure of any of claims 1 to 7, comprising several protruding elements of variable number and distribution, so that the rigidity of the structure is controlled.

9. The structure of any of claims 1 to 8, comprising openings (32) in its isolating portion.

10. An assembly comprising the structure of any of claims 1 to 9 and a rigid and/or deformable device, the structure being immovably attached to said device.
